(19) United States
(12) Patent Application Publication
(10) Pub. No.: US 2025/0382669 A1
(43) Pub. Date: Dec. 18, 2025

(54) METHOD FOR ANALYZING MODIFIED NUCLEOSIDE

(71) Applicants: SHIMADZU CORPORATION, Kyoto (JP); NATIONAL UNIVERSITY CORPORATION KUMAMOTO UNIVERSITY, Kumamoto (JP); AiSTI SCIENCE Co., Ltd., Wakayama (JP)

(72) Inventors: Akihiro KUNISAWA, Kyoto-shi, Kyoto (JP); Takanari HATTORI, Kyoto-shi, Kyoto (JP); Jun WATANABE, Kyoto-shi, Kyoto (JP); Kazuhito TOMIZAWA, Kumamoto-shi, Kumamoto (JP); Yu NAGAYOSHI, Kumamoto-shi, Kumamoto (JP); Ryoichi SASANO, Wakayama-shi, Wakayama (JP); Tomoki ASAI, Wakayama-shi, Wakayama (JP)

(73) Assignees: SHIMADZU CORPORATION, Kyoto (JP); NATIONAL UNIVERSITY CORPORATION KUMAMOTO UNIVERSITY, Kumamoto (JP); AiSTI SCIENCE Co., Ltd., Wakayama (JP)

(21) Appl. No.: 18/570,878

(22) PCT Filed: Mar. 30, 2022
(86) PCT No.: PCT/JP2022/016338

§ 371 Date: Dec. 15, 2023

(30) Foreign Application Priority Data
Jun. 15, 2021 (JP) 2021-099496

(51) Int. Cl. C12Q 1/6872

(52) U.S. Cl. CPC C12Q 1/6872

(57) ABSTRACT

An analyzing method includes: an elution step of introducing a sample containing a target component as a modified nucleoside having hydrophobicity increased by modification and a reference component as a component different from the target component into a column, and separating the target and reference components by gradient elution; a step of detecting the target and reference components by mass spectrometry; and a step of calculating a ratio between detection values of the target component and reference components. In the elution step, a mixing ratio of the solvents is changed such that between a first period in which the target component is eluted and a second period in which the reference component is eluted, a third period is provided in which a change rate of a mixing ratio at a column outlet is larger than that of a mixing ratio in each of the first and second periods.

### TECHNICAL FIELD

The present invention relates to a method for analyzing a modified nucleoside contained in a biological sample such as serum or urine.

### BACKGROUND ART

There are 22 types of transfer RNA (mt-tRNA) in human mitochondria, and it is known that these mt-tRNAs contain many chemical modifications. In recent years, the relevance between chemical modification in human mt-tRNA and disease has been pointed out, and for example, regarding taurine modification introduced into uridine at position 34 of human mt-tRNA, it has been reported that a defect of taurine modification is observed in mt-tRNA derived from a tissue collected from a mitochondrial disease patient.

Therefore, it has been investigated to analyze nucleosides of mt-IRNA, and to use the results of analyzing the presence or absence of chemical modification including taurine modification and the amount of modified nucleoside for diagnosis of mitochondrial diseases. For example, Patent Literature 1 describes a method for analyzing a biological sample such as urine and serum collected from a subject for diagnosis of mitochondrial disease using liquid chromatography (LC) or liquid chromatography-mass spectrometry (LC/MS), and measuring the amount of modified nucleoside contained in the sample. The method described in Patent Literature 1 has been made based on the finding that the number of modified nucleosides derived from mt-tRNA contained in urine, serum, and the like is significantly larger in patients with mitochondrial diseases than in healthy subjects.

### CITATION LIST

### Patent Literature

Patent Literature 1: WO 2018/124235A

### Non Patent Literature

Non Patent Literature 1: Wei et al., J Clin Invest. 2011; 121 (9): 3598-3608

### SUMMARY OF INVENTION

### Technical Problem

When the amount of a target component in a biological sample is measured by using LC or LC/MS, it is common to detect a component to be an internal standard of the biological sample (hereinafter, referred to as reference component) together with the target component, and represent the amount of the target component by a ratio between a detection value of the reference component and a detection value of the target component. This allows accurate measurement of the amount (concentration) of the target component in the sample while suppressing the influence of concentration change of the biological sample itself, deterioration of the biological sample, pretreatment, and the like on the analysis results of the target component. For example, if the biological sample is urine, creatinine is used as a reference component to suppress the influence of urine concentration, and if the biological sample is serum, a metabolic precursor of a target component is used as a reference component.

If a plurality of components contained in a biological sample are analyzed by LC/MS, there is generally used a method of using a mixed solution of a plurality of solvents having different elution forces as a mobile phase, and using gradient elution in which a mixing ratio of the solvents is continuously changed with the lapse of time to separate the plurality of components and to elute from a column.

The inventors of the present invention have found, from the results of analyzing, by LC/MS, the amount of a modified nucleoside (N⁶-threonylcarbamoyladenosine (t⁶A), 2-methylthio-N⁶-threonylcarbamoyladenosine (ms²t⁶A)) having a specific chemical structure contained in urine or a sample obtained by preparing serum collected from a subject, that it is possible to predict whether the possibility that the subject is suffering from an infectious disease called COVID-19 and a condition becomes severe is high. In addition, the modified nucleoside (t⁶A, ms²t⁶A) having a specific chemical structure described above is a modified nucleoside found to be contained in large amounts in urine and serum collected from COVID-19 patients by Tomizawa and Nagaya et al., inventors of the present invention.

However, it has been found that the modified nucleoside has increased its hydrophobicity by having a specific chemical structure (that is, by chemical modification) as compared with a nucleoside not having such a chemical structure (that is, a nucleoside not chemically modified). A general reference component in the case of analyzing components contained in urine and serum by LC/MS has high hydrophilicity, and thus it takes time to separate the modified nucleoside and the reference component described above by LC. If the LC separation takes time, the time for obtaining the amount of the modified nucleoside and the amount of the reference component becomes longer accordingly, causing such a problem that it takes time to diagnose COVID-19 or predict the severity.

Herein, the case where the modified nucleoside useful for diagnosis or prediction of severity of COVID-19 is analyzed by LC/MS has been described as an example, but a similar problem can occur if the modified nucleoside with the hydrophobicity increased by modification is measured by LC/MS together with the reference component.

The problem to be solved by the present invention is to shorten the time for correctly measuring the amount of a specific modified nucleoside contained in a sample by LC/MS.

### Solution to Problem

A method for analyzing a modified nucleoside according to the present invention, which has been made to solve the above problems, includes:
an elution step of introducing a sample containing a target component as a modified nucleoside having hydrophobicity increased by modification and a reference component as a component different from the target component into a column for liquid chromatography, and separating the target component and the reference component from each other by gradient elution in which a mixing ratio of a plurality of solvents constituting a mobile phase is changed with time to elute from the column;
a step of detecting each of the target component and the reference component by mass spectrometry; and
a step of calculating a ratio between the detection value of the target component and the detection value of the reference component, wherein
in the elution step,
a mixing ratio of the solvents constituting the mobile phase to be introduced into the column is changed such that between a first period in which the target component is eluted from the column and a second period in which the reference component is eluted from the column, a third period is provided in which a change rate of a mixing ratio of the solvents constituting the mobile phase at an outlet of the column is larger than the change rate of a mixing ratio of the solvents constituting the mobile phase in each of the first period and the second period.

### Advantageous Effects of Invention

In the method for analyzing a modified nucleoside of the present invention, in the step of separating and eluting the target component and the reference component from the LC column, between the first period and the second period, there is provided the third period in which the change rate of the mixing ratio of the solvents constituting the mobile phase at the column outlet is larger than the change rates in the first and second periods, which allows to shorten the time for separating and eluting the target component and the reference component from the column in the elution step. Therefore, the time for correctly measuring the amount of the target component contained in the sample using LC/MS can be shortened.

### BRIEF DESCRIPTION OF DRAWINGS

FIG. 1 is a view illustrating structures of t⁶A and ms²t⁶A.

FIG. 2 is a gradient schedule in LC/MS/MS analysis of First Example.

FIG. 3 is an MRM chromatogram of a sample.

FIG. 4 is a gradient schedule in LC/MS/MS analysis of Second Example.

FIG. 5A illustrates a vicinity of an acp³U peak, FIG. 5B illustrates a vicinity of a t⁶A peak, and FIG. 5C illustrates a vicinity of a ms²t⁶A peak in an MRM chromatogram of a sample.

FIG. 6 illustrates the analysis result of Third Example, and is a graph illustrating the amount of t⁶A as the target component contained in the serum of COVID-19 patients and healthy subjects, as a ratio to the measured value of adenosine as the reference component.

FIG. 7 illustrates the analysis result of Third Example, and is a graph illustrating the amount of t⁶A as the target component contained in the serum of COVID-19 patients and healthy subjects, as a measured value of t⁶A.

FIG. 8 illustrates the analysis result of Fourth Example, and is a graph illustrating the amount of ms²t⁶A as the target component contained in the serum of COVID-19 patients and healthy subjects, as a ratio to the measured value of adenosine as the reference component.

FIG. 9 illustrates the analysis result of Fourth Example, and is a graph illustrating the amount of ms²t⁶A as the target component contained in the serum of COVID-19 patients and healthy subjects, as a measured value of ms²t⁶A.

### DESCRIPTION OF EMBODIMENTS

A method for analyzing a modified nucleoside of the present invention is a method for analyzing, by LC/MS, the amount of a target component in a sample using, as a target component, a modified nucleoside having hydrophobicity increased particularly by modification among the modified nucleosides, and using a component different from the target component as a reference component. In the present invention, calculating the ratio between the detection value of the target component and the detection value of the reference component allows the amount of the target component to be correctly obtained.

In the present invention, it is preferable that a modified nucleoside having a predetermined chemical structure among the modified nucleosides contained in the sample is used as a target component, and a component that is contained in the sample and does not have the predetermined chemical structure is used as a reference component. A modified nucleoside having a predetermined chemical structure has increased hydrophobicity than a nucleoside not having the predetermined chemical structure (unmodified nucleoside). Therefore, the predetermined chemical structure is a structure that increases hydrophobicity of the modified nucleoside. The reference component may be a modified nucleoside or an unmodified nucleoside as long as it does not have the predetermined chemical structure. Alternatively, other components may be used.

The sample used in the method for analyzing a modified nucleoside of the present invention is typically a biological sample such as serum, plasma, or urine collected from a mammal (particularly, human), and may be a sample obtained by subjecting the biological sample to a predetermined pretreatment. If the biological sample possibly containing a modified nucleoside is urine, creatinine is generally used as a reference component, and if it is blood (whole blood, plasma, serum), a metabolic precursor of the target component is generally used as a reference component. All of these reference components are known to be highly hydrophilic (less hydrophobic).

The predetermined chemical structure is, of course, a structure that affects the hydrophobicity of the modified nucleoside, and is preferably a structure that is likely to affect the strength of retention of the target component and the reference component on the column of the LC. In this case, the target component having such a chemical structure has such a property that the target component is easily retained by the column (strong retention strength) or is hardly retained by the column (weak retention strength) as compared with the reference component not having the chemical structure.

In the method for analyzing a modified nucleoside of the present invention, the target component and the reference component are separated from each other and eluted from the column by gradient elution. If the target component has a stronger retention strength on the column than the reference component, a first period in which the target component is eluted from the column is later than a second period in which the reference component is eluted, and if the target component has a weaker retention strength than the reference component, the first period is earlier than the second period.

In the period between the first period and the second period, a component that is neither the target component nor the reference component, that is, a component unnecessary for measuring the amount of the target component (unnecessary component or contaminant component) is eluted from the column. In the present invention, between the first period and the second period, a third period is provided in which the change rate of the mixing ratio of the solvent of the mobile phase at the column outlet is larger than the change rate of the mixing ratio of the solvent of the mobile phase at the column outlet in each of the first period and the second period, and thus it is possible to shorten the time during which the unnecessary component or the contaminant component is eluted from the column. This allows the time for correctly measuring the amount of the target component using LC/MS to be shortened as a whole.

The change rate of the mixing ratio of the solvent in the mobile phase in each of the first to third periods may be constant or need not be constant. If the change rate of the mixing ratio of the solvent of the mobile phase in each period is not constant, the average change rate (average value of the change rate) can be defined as the change rate in the period. In addition, the third period and each of the first period and the second period need not be strictly separated, and a part or all of the third period may overlap the first period and/or the second period.

In the method for analyzing a modified nucleoside according to the present invention, an elution step is performed according to a gradient program in which the change rate of the mixing ratio of the solvent of the mobile phase at the column outlet in the first to third periods has the above-described relationship. Such a gradient program can be determined by calculating the time required from a solvent mixing part of the mobile phase to the column outlet on the basis of, for example, the length of the flow path from the solvent mixing part of the mobile phase to the column, the length, type, and characteristics of the column, and other factors affecting the flow rate of the mobile phase, and adjusting the timing of changing the mixing ratio in the solvent mixing part of the mobile phase so that the target component and the reference component are eluted from the column outlet at a desired timing. In this case, a plurality of change rates of the mixing ratio of the solvent of the mobile phase may be prepared, the respective change rates of the mixing ratio of the solvent of the mobile phase at the column outlet when the mobile phase is introduced into the column at the plurality of change rates may be determined, and the gradient program may be determined based on the relationship between the change rate of the mixing ratio of the solvent of the mobile phase at the column inlet and the change rate of the mixing ratio of the solvent of the mobile phase at the column outlet.

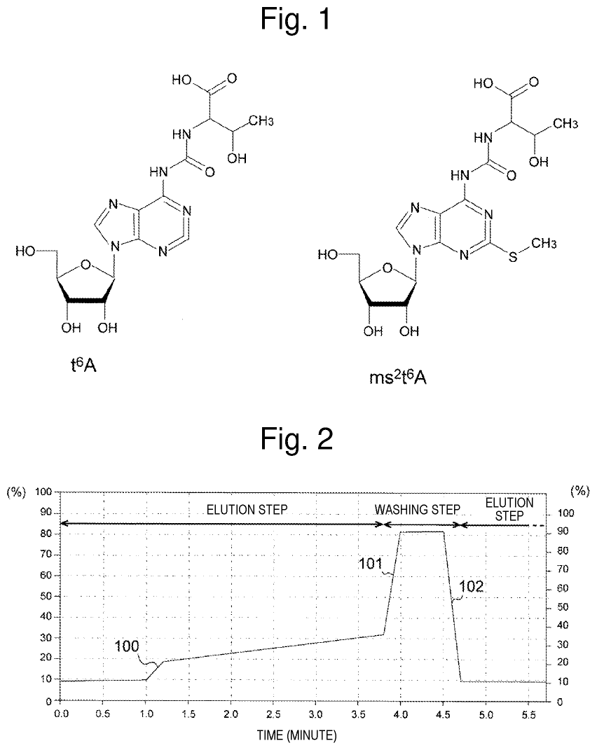

If the column of LC is a column for reverse phase chromatography, examples of the chemical structure that are likely to affect the retention strength on the column include a chemical structure in which threonine is bound to position 6 of adenosine via a carbonyl group, and as a modified nucleoside having such a chemical structure, there are N⁶-threonylcarbamoyladenosine (t⁶A), which is a modified adenosine derived from mitochondrial IRNA (mt-tRNA), and 2-methylthio-Ne-threonylcarbamoyladenosine (ms²t⁶A), which is a modified adenosine derived from cytoplasmic tRNA. FIG. 1 illustrates structures of t⁶A and ms²t⁶A.

t⁶A is a modified base present at position 37 of the IRNA that decodes the ANN codon, and is a modified nucleoside that is conserved in almost all organisms and is essential for the growth of many organisms. t⁶A is known to play important roles in various stages of protein synthesis, such as aminoacylation of IRNA, translocation reactions, accurate recognition of codons, and maintenance of reading frames.

ms²t⁶A has a chemical structure in which position 2 of adenine of t⁶A is thiomethylated. It has been reported by the inventors of the present invention that ms²t⁶A is a modified base present at position 37 of IRNA whose anticodon is UUU, and is biosynthesized from t⁶A by Cdkal1, which is a methylthiotransferase (Non Patent Literature 1).

In addition, using the method of the present invention allows the amounts of t⁶A and ms²t⁶A in the blood and urine of a subject to be accurately measured in a short time. Therefore, comparing the amounts of t⁶A and ms²t⁶A in serum and urine with the index value indicating possibility that the subject is suffering from COVID-19 and/or the index value indicating possibility that the subject suffers from COVID-19 and a condition becomes severe allows diagnosis on whether or not the subject is suffering from and prediction of the severity in the case of suffering from COVID-19 at an early stage with high reliability. Therefore, the method for analyzing a modified nucleoside of the present invention can be said to be a useful method for early diagnosis and prediction of severity of COVID-19.

Hereinafter, the present invention is described in detail based on Examples, but these Examples do not limit the present invention at all.

### First Example

First, to examine whether or not it is possible to measure a modified nucleoside having a specific chemical structure contained in a biological sample by multiple reaction monitoring (MRM) measurement using a liquid chromatograph mass spectrometer (LC/MS/MS), standard samples of target components and reference components were dissolved in water to prepare samples, and the samples were subjected to MRM measurement. The standard samples used for the measurement were as follows.

### <Target Components>

N⁶-Threonylcarbamoyladenosine (t⁶A) (manufactured by Cosmo Bio Co., Ltd.)
2-Methylthio-N⁶-threonylcarbamoyladenosine (ms²t⁶A) (manufactured by Santa Cruz Biotechnology, Inc.)

### <Reference Components>

3-Amino-3-carboxypropyluridine (acp³U) (manufactured by Carbosynth Inc.)
Adenosine (manufactured by Sigma-Aldrich Japan Co. LLC.)

acp³U is one of the modified nucleosides. Adenosine is one of nucleosides.

In addition, the apparatus names and analysis conditions used for the measurement are as follows. In the present specification, “%” represents % by volume (% (v/v)).

### <Apparatuses>

Liquid chromatograph: Ultra high performance liquid chromatograph Nexera X3 (manufactured by Shimadzu Corporation)
Mass spectrometer: Ultra high performance triple quadrupole mass spectrometer LCMS-8060 (manufactured by Shimadzu Corporation)

### <LC Analysis Conditions>

Column: Mastro2 C18 (inner diameter 2.1 mm×length 150 mm, particle diameter 3 μm, manufactured by Shimadzu GLC Ltd.)
Mobile phase A: 0.1% formic acid-water
Mobile phase B: 0.1% formic acid-acetonitrile
Gradient: Mobile phase B concentration: 10% (0 to 1.0 min)→20% (1.2 min)→35% (3.8 min)→90% (4.0 to 4.5 min)→10% (4.7 to 5.7 min)
Flow rate: 0.3 mL/min
Column temperature: 40° C.
Injection volume: 2 μL

### <MS Analysis Conditions>

Ionization mode: ESI
Nebulizer gas flow rate: 3.0 L/min
Drying gas flow rate: 10 L/min
Heating gas flow rate: 10 L/min
Interface temperature: 300° C.
DL temperature: 250° C.
Heat block temperature: 400° C.

FIG. 2 is a graph illustrating a temporal change in a mixing ratio of a solvent constituting a mobile phase (hereinafter, also referred to as “mixing ratio of mobile phase”) in gradient elution. In addition, the retention time of the target components and the reference components under the LC analysis conditions, and the MRM transitions of the quantitative ions and the confirmation ions are listed in Table 1. In Table 1, “Polarity” represents the polarity of the ions to be measured.

TABLE 1

| | | Retention time | | Quantitative ion | Confirmation ion |
|---|---|---|---|---|---|
| No. | Compound name | (min) | Polarity | (m/z) | (m/z) |
| 1 | acp3U | 1.75 | + | 346.10 > 214.00 | − |
| 2 | 16A | 3.95 | + | 413.10 > 281.10 | − |
| 3 | ms216A | 4.10 | + | 459.10 > 327.10 | 459.10 > 208.10 |

FIG. 2 illustrates the temporal change in the mixing ratio of the mobile phase when the mobile phase is introduced into the column. As illustrated in FIG. 2, in the present example, the components retained in the column are eluted by the mobile phase introduced into the column in the period from time 0 min to 3.80 min, and the column is washed by the mobile phase introduced into the column in the period from 3.80 min to 4.7 min.

When the mobile phase is introduced into the column, the components are eluted from the column while the mobile phase moves from the inlet to the outlet of the column, and the column is washed. Therefore, the step of eluting the component from the column and the step of washing the column are shifted backward in time from the timing at which the mobile phase is introduced into the column. However, in the following description, for convenience, the step of introducing the mobile phase involved in the elution step and the washing step into the column are referred to as elution step and washing step, respectively.

In the present example, the mixing ratio of the mobile phase rapidly changes in a period from time 1.0 min to 1.2 min in the elution step (a period denoted by reference numeral 100 in FIG. 2), and the periods before and after this period 100 include a second period in which the reference components (acp³U, adenosine) are eluted from the column and a first period in which the target components (t⁶A and ms²t⁶A) are eluted from the column, respectively. That is, the period 100 corresponds to a third period in which the change rate of the mixing ratio of the mobile phase is larger than the change rate of the mixing ratio of the mobile phase in each of the first period and the second period. In addition, the mixing ratio of the mobile phase rapidly changes in the early and late periods (time period 101 from 3.8 min to 4.0 min and period 102 from 4.5 min to 4.7 min) in the washing step, and the change rate of the mixing ratio is larger than the change rate of the mixing ratio of the mobile phase in each of the first period and the second period. Therefore, these periods 101 and 102 correspond to a fourth period of the present invention.

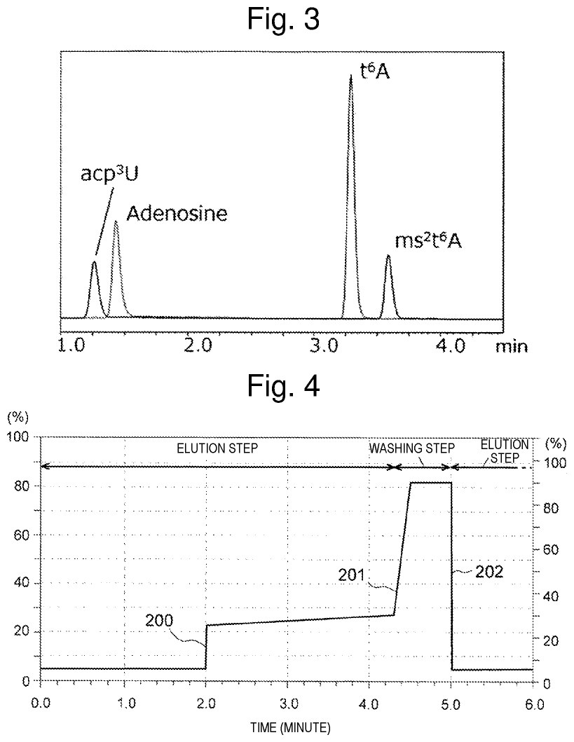

FIG. 3 illustrates an MRM chromatogram obtained for a sample containing acp³U, adenosine, t⁶A, and ms²t⁶A. From FIG. 3, as a result of analysis under the analysis conditions of the present example, it has been found that two reference components and two target components contained in the sample can be separated and eluted from the column, and each component can be individually analyzed without mutual interference.

As is found from FIG. 2, in the present example, the time taken for the target components and the reference components contained in the sample to be eluted from the column was 4.7 minutes including the elution step and the washing step. In addition, as is found from FIG. 3, the reference components were eluted in a period of time from 1.25 min to 1.55 min, and the target components were eluted in a period of time from 3.20 min to 3.75 min. In practice, it took 2.50 min for all of the reference components and the target components to be eluted from the column. It has been found that the measurement time of the target components can be significantly shortened in the method of the present example, in which the third period and the fourth period are provided, as compared with the conventional method, in which the elution step and the washing step together take 30 minutes or more.

### Second Example

A multiple reaction monitoring (MRM) measurement using a liquid chromatograph mass spectrometer (LC/MS/MS) was performed on a biological sample collected from a subject under the same conditions as in First Example except that the condition of gradient elution in LC analysis was changed. In the present example, t⁶A and ms²t⁶A were used as target components, and acp³U was used as a reference component.

As the biological sample used for the analysis, serum collected from a patient diagnosed as suffering from COVID-19 was used.

The apparatus names and analysis conditions used for the analysis are as follows.

### <Apparatuses>

Liquid chromatograph: Ultra high performance liquid chromatograph Nexera X2 (manufactured by Shimadzu Corporation)
Mass spectrometer: Ultra high performance triple quadrupole mass spectrometer LCMS-8045 (manufactured by Shimadzu Corporation)

### <LC Analysis Conditions>

Column: Mastro2 C18 (inner diameter 2.1 mm×length 150 mm, particle diameter 3 μm, manufactured by Shimadzu GLC Ltd.)
Mobile phase A: 0.1% formic acid-water
Mobile phase B: 0.1% formic acid-acetonitrile
Gradient: Mobile phase B concentration: 5% (0 to 2.0 min)→25% (2.01 min)→30% (4.3 to 4.5 min)→90% (4.5 to 5.0 min)→5% (5.01 to 6.0 min)
Flow rate: 0.3 mL/min
Column temperature: 40° C.
Injection volume: 2 μL

### <MS Analysis Conditions>

Ionization mode: ESI
Nebulizer gas flow rate: 3.0 L/min
Drying gas flow rate: 10 L/min
Heating gas flow rate: 10 L/min
Interface temperature: 300° C.
DL temperature: 250° C.
Heat block temperature: 400° C.

FIG. 4 is a graph illustrating a temporal change in the mixing ratio of the mobile phase in gradient elution. In addition, the retention time of the target components and the reference component under the LC analysis conditions, and the MRM transitions of the quantitative ions and the confirmation ions are listed in Table 2. In Table 2, “Polarity” represents the polarity of the ions to be measured.

TABLE 2

| | | Retention time | | Quantitative ion | Confirmation ion |
|---|---|---|---|---|---|
| No. | Compound name | (min) | Polarity | (m/z) | (m/z) |
| 1 | acp3U | 1.273 | + | 346.10 > 214.00 | − |
| 2 | adenosine | 1.428 | + | 268.10 > 136.05 | 268.10 > 119.00 |
| 3 | 16A | 3.293 | + | 413.10 > 281.10 | − |
| 4 | ms216A | 3.591 | + | 459.10 > 327.10 | 459.10 > 208.10 |

Similarly to FIG. 2, FIG. 4 illustrates the temporal change in the mixing ratio of the mobile phase when the mobile phase is introduced into the column. As illustrated in the graph of FIG. 4, in the present example, a period from time 0 min to 4.30 min corresponds to the elution step, and a period from time 4.30 min to 5.00 min corresponds to the washing step. The mixing ratio of the mobile phase rapidly changes during a period from time 2.0 min to 2.01 min in the elution step (a period denoted by reference numeral 200 in FIG. 4), and the periods before and after this period 200 include a second period in which the reference component (acp³U) is eluted from the column and a first period in which the target components (t⁶A and ms²t⁶A) are eluted from the column, respectively. This period 200 corresponds to a third period of the present invention. In addition, in the washing step, the mixing ratio of the mobile phase rapidly changes in a period 201 from time 4.30 min to 4.50 min and a period 202 from time 5.0 min to 5.01 min, and these periods 201 and 202 correspond to a fourth period of the present invention.

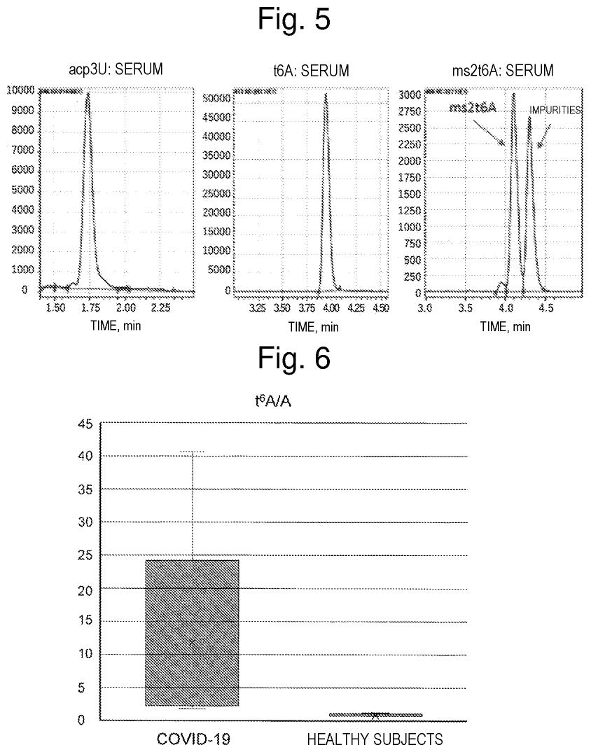

FIG. 5 illustrates MRM chromatograms obtained for acp³U, t⁶A, and ms²t⁶A. As is found from FIG. 5, as a result of analysis under the analysis conditions of the present example, it has been found that one reference component and two target components contained in the sample can be separated and eluted from the column, and each component can be individually analyzed without mutual interference. In addition, in the present example, the time taken to elute one reference component and two target components contained in the sample from the column was a sum of 5.0 minutes in the elution step and the washing step, and the time taken to actually elute all three components from the column was about 2.6 minutes (1.60 min to 4.20 min). It has been found that the measurement time of the target components can be significantly shortened in the method of the present example, in which the third period and the fourth period are provided, as compared with the conventional method, in which the elution step and the washing step together take 30 minutes or more.

### Third Example

Samples prepared from urine collected from patients diagnosed as suffering from COVID-19 and healthy subjects were subjected to multiple reaction monitoring (MRM) measurement using a liquid chromatograph mass spectrometer (LC/MS/MS) under the same conditions as in Second Example. Then, the ratios between the obtained measured values of t⁶A and ms²t⁶A and the measured value of adenosine was determined, and these values were regarded as the amount of 16A and ms²t⁶A contained in the sample.

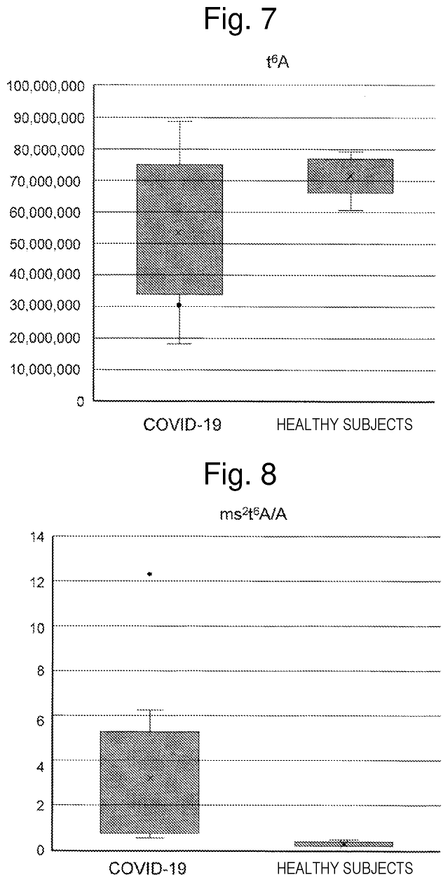

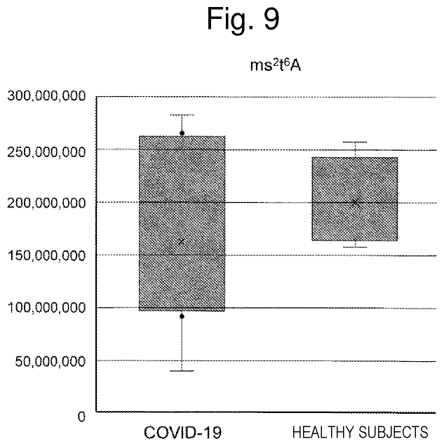

FIGS. 6 and 8 illustrate the amount of t⁶A and the amount of ms²t⁶A obtained from the ratios between the measured values of t⁶A and ms²t⁶A and the measured value of adenosine. On the other hand, FIGS. 7 and 9 illustrate the amount of 16A and the amount of ms²t⁶A obtained from the measurement values of t⁶A and ms²t⁶A (that is, without using the measured value of adenosine).

From FIGS. 6 to 9, for any of the target components t⁶A and ms²t⁶A, when the measured value was represented as a ratio to the measured value of adenosine as a reference component, a significant difference was observed in the amount of the target component contained in the serum between COVID-19 patients and healthy subjects. On the other hand, when the measured value of the reference component was not used, there was no significant difference between COVID-19 patients and healthy subjects.

In addition, when using the ratio between the measured value of the target component and the measured value of the reference component, the amount of the target component contained in the urine of healthy subjects was almost 0, and the variation was small as compared with the case where the measured value of the reference component was not used.

From the above results, the amount of the target component contained in the sample can be correctly evaluated by representing the measured value of the target component in a ratio to the measured value of the reference component, thus suggesting that using the method for analyzing a modified nucleoside of the present invention is useful for determining the possibility of suffering from COVID-19 and/or predicting the severity of COVID-19 patients.

### [Various Modes]

It is obvious for those skilled in the art that the exemplary embodiments described above are specific examples of the following modes.

(Clause 1) A method for analyzing a modified nucleoside according to the present invention includes:
an elution step of introducing a sample containing a target component as a modified nucleoside having hydrophobicity increased by modification and a reference component as a component different from the target component into a column for liquid chromatography, and separating the target component and the reference component from each other by gradient elution in which a mixing ratio of a plurality of solvents constituting a mobile phase is changed with time to elute from the column;
a step of detecting each of the target component and the reference component by mass spectrometry; and
a step of calculating a ratio between a detection value of the target component and a detection value of the reference component, wherein
in the elution step,
a mixing ratio of the solvents constituting the mobile phase to be introduced into the column is changed such that between a first period in which the target component is eluted from the column and a second period in which the reference component is eluted from the column, a third period is provided in which a change rate of a mixing ratio of the solvents constituting the mobile phase at an outlet of the column is larger than a change rate of a mixing ratio of the solvents constituting the mobile phase in each of the first period and the second period.

With the method for analyzing a modified nucleoside of Clause 1, in the step of separating and eluting the target component and the reference component from the LC column, the mixing ratio of the solvents constituting the mobile phase to be introduced into the column is changed such that between the first period and the second period, the third period is provided in which the change rate of the mixing ratio of the solvents constituting the mobile phase is larger than the change rates of the first and second periods at the outlet of the column, whereby the time for separating and eluting the target component and the reference component from the column in the elution step can be shortened. Therefore, using the detection value of the target component and the detection value of the reference component obtained by using LC/MS can shorten the time for correctly measuring the amount of the target component contained in the sample. Although the component in the sample is eluted from the column also in the third period, this component can be said to be a component that is not necessary for measuring the amount of the target component contained in the sample, and thus increasing the change rate of the mixing ratio of the solvent in the mobile phase in the third period does not adversely affect the correct measurement of the amount of the target component in the sample.

(Clause 2) In the method for analyzing a modified nucleoside of Clause 1, the target component may be a derivative of adenosine, may be a modified nucleoside having a chemical structure in which threonine is bonded to the adenosine via a carbonyl group, and the reference component may be a component not having the chemical structure.

With the method for analyzing a modified nucleoside of Clause 2, using a property of the chemical structure can relatively easily adjust an interval between elution time of the target component and elution time of the reference component.

(Clause 3) In the method for analyzing a modified nucleoside of Clause 1,
the target component may be N⁶-threonylcarbamoyladenosine and/or 2-methylthio-N⁶-threonylcarbamoyladenosine, and
the method may further include a step of comparing an obtained amount of the target component with at least one of an index value indicating possibility that a subject from which the sample is collected is suffering from COVID-19 and an index value indicating possibility that the subject suffers from COVID-19 and a condition becomes severe.

With the method for analyzing a modified nucleoside of Clause 3, there can be determined, in a short time, whether or not there is a possibility that the subject from which the sample is collected is suffering from COVID-19 or a possibility that a condition of the subject suffering from COVID-19 becomes severe, based on a comparison result between the amount of the target component and the index value.

(Clause 4) In the method for analyzing a modified nucleoside of any one of Clauses 1 to 3,
the sample may be urine, and
the reference component may be at least one selected from the group consisting of creatinine, urea nitrogen, uric acid, adenosine, and 3-amino 3-carboxypropyluridine.

With the method for analyzing a modified nucleoside of Clause 4, an influence of a concentration of urine on an analysis result of the target component can be suppressed.

(Clause 5) In the method for analyzing a modified nucleoside of any one of Clauses 1 to 3,
the sample may be plasma or serum, and
the reference component may be at least one of adenosine and 3-amino 3-carboxypropyluridine.

With the method for analyzing a modified nucleoside of Clause 5, the influence of the time until the plasma or serum collected from the subject is subjected to analysis or the pretreatment performed on the plasma or serum on the analysis result of the target component can be suppressed.

(Clause 6) In the method for analyzing a modified nucleoside of any one of Clauses 1 to 5,
the mobile phase may be a mixed solution of formic acid, acetonitrile, and water.

(Clause 7) In the method for analyzing a modified nucleoside of any one of Clauses 1 to 6,
the liquid chromatography may be reverse phase chromatography.

With the method for analyzing a modified nucleoside of Clause 6 or 7, the target component and the reference component can be reliably separated and eluted from the column.

(Clause 8) In the method for analyzing a modified nucleoside of any one of Clauses 1 to 7,
the method may include a washing step of washing the column using the mobile phase after the elution step, and
in the washing step, the mixing ratio of the solvents constituting the mobile phase to be introduced into the column may be changed such that in an early stage or a late stage of the washing step, a fourth period is provided in which a change rate of the mixing ratio of the solvents constituting the mobile phase is larger than a change rate of the mixing ratio of the solvents constituting the mobile phase in each of the first period and the second period.

With the method for analyzing a modified nucleoside of Clause 8, the time required for transition from the elution step to the washing step or transition from the washing step to the subsequent elution step can be shortened.

1. A method for analyzing a modified nucleoside, the method comprising:
an elution step of introducing a sample containing a target component as a modified nucleoside having hydrophobicity increased by modification and a reference component as a component different from the target component into a column for liquid chromatography, and separating the target component and the reference component from each other by gradient elution in which a mixing ratio of a plurality of solvents constituting a mobile phase is changed with time to elute from the column;
a step of detecting each of the target component and the reference component by mass spectrometry; and
a step of calculating a ratio between a detection value of the target component and a detection value of the reference component, wherein
in the elution step,
a mixing ratio of the solvents constituting the mobile phase to be introduced into the column is changed such that between a first period in which the target component is eluted from the column and a second period in which the reference component is eluted from the column, a third period is provided in which a change rate of a mixing ratio of the solvents constituting the mobile phase at an outlet of the column is larger than a change rate of a mixing ratio of the solvents constituting the mobile phase in each of the first period and the second period.

2. The method for analyzing a modified nucleoside according to claim 1, wherein the target component is a derivative of adenosine, is a modified nucleoside having a chemical structure in which threonine is bonded to the adenosine via a carbonyl group, and the reference component is a component not having the chemical structure.

3. The method for analyzing a modified nucleoside according to claim 2,
wherein the target component is N⁶-threonylcarbamoyladenosine and/or 2-methylthio-N⁶-threonylcarbamoyladenosine,
the method further comprising a step of comparing an obtained amount of the target component with at least one of an index value indicating possibility that a subject from which the sample is collected is suffering from COVID-19 and an index value indicating possibility that the subject suffers from COVID-19 and a condition becomes severe.

4. The method for analyzing a modified nucleoside according to claim 1, wherein
the sample is urine, and
the reference component is at least one selected from the group consisting of creatinine, urea nitrogen, uric acid, adenosine, and 3-amino 3-carboxypropyluridine.

5. The method for analyzing a modified nucleoside according to claim 1, wherein
the sample is plasma or serum, and
the reference component is at least one of adenosine and 3-amino 3-carboxypropyluridine.

6. The method for analyzing a modified nucleoside according to claim 1, wherein the mobile phase is a mixed solution of formic acid, acetonitrile, and water.

7. The method for analyzing a modified nucleoside according to claim 1, wherein the liquid chromatography is reverse phase chromatography.

8. The method for analyzing a modified nucleoside according to claim 1, comprising a washing step of washing the column using the mobile phase after the elution step, wherein
in the washing step, a mixing ratio of the solvents constituting the mobile phase to be introduced into the column is changed such that in an early stage or a late stage of the washing step, a fourth period is provided in which a change rate of a mixing ratio of the solvents constituting the mobile phase is larger than a change rate of a mixing ratio of the solvents constituting the mobile phase in each of the first period and the second period.
